(19) United States
(12) Patent Application Publication
(10) Pub. No.: US 2012/0042611 A1
(43) Pub. Date: Feb. 23, 2012

(54) Apparatus for Treating Packagings

(75) Inventor: Ulrich LAPPE, Regensburg (DE)

(73) Assignee: KRONES AG, Neutraubling (DE)

(21) Appl. No.: 13/211,293

(22) Filed: Aug. 16, 2011

(30) Foreign Application Priority Data
Aug. 19, 2010 (DE) 10 2010 034 895.3

(51) Int. Cl. B65B 55/10

(52) U.S. Cl. 53/426; 422/243

(57) ABSTRACT

An apparatus for treating packagings, and in particular containers, may include at least one transport unit which transports the containers along a specified transport path. The transport unit includes a transport element which contacts at least an area of the containers during transport via a first contact surface. The apparatus includes at least one treatment unit which treats the containers in a specified manner. The treatment unit has a treatment element which is in contact, at least temporarily, with a surface of the container via a second contact surface. At least one of the first and second contact surfaces is designed to be germicidal.

### CROSS-REFERENCE TO RELATED APPLICATION

This application claims the benefit of priority of German Patent Application No. 10 2010 034 895.3, filed Aug. 19, 2010, pursuant to 35 U.S.C. 119(a)-(d), the disclosure of which is incorporated herein by reference.

### TECHNICAL FIELD

The present disclosure relates to an apparatus for treating packagings, in particular containers such as for example preforms or containers.

### BACKGROUND

Such apparatus have been known from the prior art for a long time. These apparatus may for example be apparatus that mould plastic preforms into plastic containers, such as for example blow moulding machines. These may be sterilisation devices which sterilise containers or plastic preforms, transport units for containers or preforms and the like. The subject of the disclosure is described in particular with reference to such apparatus which mould plastic preforms into plastic containers. In this connection it is known from the prior art that the sterilisation units sterilise the plastic preforms or the containers. After this process, the plastic preform will have less germs and/or will be free of product pests. However, in the subsequent blowing process and as a result of contact with the machine parts, such as for example during transfers, but also with the stretching rod that stretches the plastic preforms, or with the blow nozzles, there is a risk that the plastic preforms or the containers will be re-contaminated with product pests.

It may therefore be desirable to provide an apparatus for reducing the risk of such re-contaminations.

### SUMMARY

An apparatus according to the disclosure for treating packagings, and in particular containers, comprises a transport unit that transports the containers along a specified transport path. This transport unit includes a transport member which contacts at least an area of the container during transport over a first contact surface. Further, a treatment unit is provided which treats the containers in a specified manner, said treatment unit including a treatment member which is in contact, at least temporarily, with the surface of the container via a second contact surface. According to the disclosure, at least one of said contact surfaces is designed or modified to be germicidal. The term germicidal is to be understood to mean any reduction of microorganisms, in particular product pests, as well as a reduction of the reproduction thereof. In some aspects, several contact surfaces and especially preferably all contact surfaces which are in contact, at least temporarily, with the surface of the container, are designed or modified to be germicidal.

A germicidal design or modification is to be understood to mean that said surface has been subjected in particular to a treatment that modified it in such a way that any germs that get onto this surface will be killed or generally rendered harmless or their reproduction is prevented.

In general, all machine parts that come into contact with the plastic containers or the plastic preforms should be modified on the surface in such a way that they have a germicidal effect.

Plastic containers are to be understood to mean here not only the actual containers, but also their components such as in particular the closure caps thereof. Thus, the subject of the disclosure can also be applied to such contact surfaces which are in contact with a surface of the container closure caps and in particular with the internal surface of the closure caps. Corresponding apparatus may for example be a capper that closes the containers with closure caps. The treatment member may here be a capper head that receives the container closure cap and for example screws the closure cap onto the container. The transport unit may be a transport unit for transporting closure caps.

In an exemplary embodiment, at least one contact surface has a germicidal coating. Here, for example one surface may include a silver ion coating. The use of silver ion coatings has already been described in the prior art. Thus for example DE 10 2007 025 452 A1, the content of which is herewith included by reference in the subject matter of the present application in its entire scope, describes a method for coating surfaces with micro and nanoparticles using a plasma process.

In an exemplary embodiment, the apparatus includes a cleanroom, within which the containers are transported. Thus, it is possible for the containers to be filled with a beverage in this cleanroom, for plastic preforms to be moulded into the containers in this cleanroom, if required even for the plastic preforms to be heated in said cleanroom before they are moulded in a subsequent blowing machine. This cleanroom or sterile room is used here to carry out the corresponding container treatment under sterile conditions.

In an exemplary embodiment, the transport member is selected from a group of transport members such as grippers, heating mandrels, sorting rollers, discs of a disc sorter and the like.

In some aspects, the transport unit includes a plurality of transport members which transport a plurality of containers at the same time, with the containers preferably being transported separated from each other.

Further, these may also be transport members which contact the container by its mouth area for transporting it, such as for example grippers that engage below a support ring of the plastic preform or the plastic container.

In an exemplary embodiment, the treatment member is selected from a group of treatment members including blow nozzles, stretching rods, blow moulds, heating mandrels, support rings and the like. Thus, in many apparatus, a blow mould contacts for example a mouth area of the containers and for this reason at least this contact area with the mouth of the container is treated to be germicidal. In the case of the stretching rods it is in particular possible for the end section or the tips thereof to contact the internal wall of the plastic preform, so that, in some aspects, at least the tips are modified so as to be germicidal. The support rings can, for example, support the plastic preforms during the expansion thereof in relation to a blow mould. The blow moulds contact in particular an external wall of the plastic preform during the expansion thereof.

In an exemplary embodiment, the apparatus includes a sterilisation device that sterilises the containers at least in sections. It is possible here to apply a sterilisation agent onto the containers, however, sterilising the containers for example by means of electron beams (E-beams) or by means of UV radiation and the like would also be conceivable. Combinations of several sterilisation methods would also be conceivable. As a sterilisation agent, for example hydrogen peroxide or also peracetic acid could be used here.

In an exemplary embodiment, at least one contact area is arranged downstream with regard to said sterilisation unit in the transport direction of the containers. This means that upon sterilisation of the containers or of the plastic preforms, a renewed re-contamination of the containers is avoided. Thus, it is possible that after said sterilisation unit, the containers are contacted only via contact surfaces that have said germicidal effect. This may for example be carried out until the containers have been filled and closed with a closure cap.

In an exemplary embodiment, the apparatus includes a moulding device for moulding plastic preforms into plastic containers.

In an exemplary embodiment, the apparatus may also have a system for filling containers with a liquid and in particular with a beverage.

In an exemplary embodiment, all the contact surfaces that come into contact with the containers have been modified in a specified transport area of the containers so as to be germicidal.

In an exemplary embodiment, at least one contact surface is a contact surface that contacts an internal wall of the containers or a thread area of the plastic preform. This applies in particular to certain grippers or may apply to the above-mentioned stretching rod or to the blow nozzles.

The present disclosure is further directed to a method for treating packagings and in particular containers, wherein the containers are transported by means of transport members of a first transport unit and are treated using treatment members of a treatment unit, and wherein the transport members are in contact, at least temporarily, with the containers in a first contact surface, and the treatment members of the treatment unit are temporarily in contact with the container by a second contact surface. According to the disclosure, at least one contact surface is designed to be germicidal. A germicidal embodiment is here understood to mean in particular that there is a higher probability of any germs getting onto this contact surface to be killed or their reproduction to be prevented than on other surfaces of the element concerned.

In some aspects, the plastic containers or plastic preforms are sterilised and preferably at least those contact surfaces are designed to be germicidal which contact these containers upon the sterilisation thereof in the transport direction of the containers.

Further advantages and embodiments will become evident from the attached drawings.

### BRIEF DESCRIPTION OF THE DRAWINGS

In the drawings:

FIG. 1 shows a very schematic view of an exemplary system according to various aspects of the disclosure; and

FIG. 2 shows a view for illustrating the exemplary contact surfaces according to various aspects of the disclosure.

### DETAILED DESCRIPTION

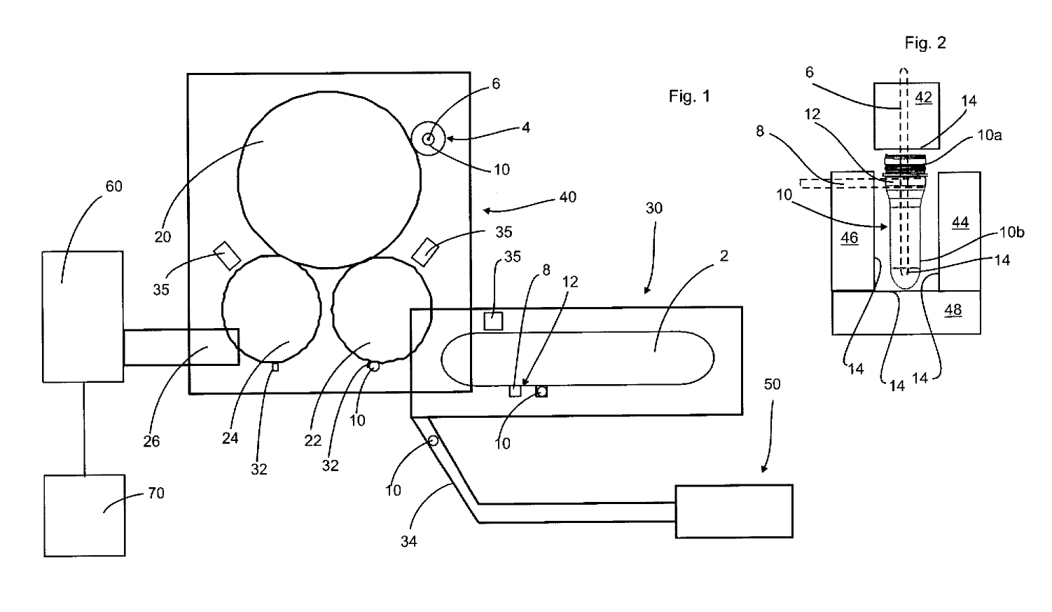

FIG. 1 shows a very schematic view of an apparatus 1 according to the disclosure. This apparatus 1 includes a heating unit 30 through which plastic preforms 10 pass whilst being heated. The heating unit 30 includes a transport unit 2 and a plurality of transport members 8 which transport the plastic preforms 10 in a separated manner. These transport members may for example be mandrels which engage in a mouth of the plastic containers 10. The external wall of these mandrels is in this case the contact surface 12 with the plastic preforms. Apart from that, the transport members may also be grippers that engage in the plastic preforms below the support ring thereof. In this case, the internal surfaces of these grippers which contact the plastic preforms may be modified so as to be germicidal. As a transport unit, for example a transport chain may be provided on which a plurality of transport members is arranged.

After passing through the heating unit 30, the plastic preforms are transferred via a further transport unit 22 to a moulding unit 40, more specifically, a blow wheel 20 of this moulding unit 40. This blow wheel 20 (which is rotatable about a specified rotary axis) has here a plurality of treatment units 4 (only one is shown) such as for example blow stations, within which the plastic preforms 10 are expanded into plastic containers by the application of pressurised air.

Reference numeral 6 relates to a treatment member such as for example a stretching rod which stretches the plastic preforms in the longitudinal direction thereof. After the moulding process, the plastic preforms are transported by a further transport unit 24 that includes a plurality of transport members 32.

Reference numeral 26 relates to a further transport unit which transfers the moulded containers to a filling unit 60 (only schematically shown). An element of this filling unit 60, such as for example a filling element, may here also have contact surfaces that are designed to be germicidal. The filling unit is followed by a closing unit 70 (also shown only schematically) which closes the plastic preforms with closure caps. Here, the surfaces of transport units that transport these closure caps may also be modified so as to be germicidal. Also treatment elements such as closure caps may be modified in a corresponding manner as mentioned above so as to be germicidal.

Reference numeral 50 relates to a reservoir which provides plastic preforms 10. The plastic preforms may be conveyed for example via a transport unit 34 such as a roll sorter. It would be possible here if the surfaces of the rollers of this roll sorter 34 were also designed to be germicidal.

In addition to or instead of the reservoir, also a moulding machine for producing the plastic preforms may be provided. Further, the system shown additionally has a cleanroom through which the plastic preforms are transported. Therefore, it would be possible here for only individual areas of the system to have a corresponding cleanroom, for example all the system parts from the moulding unit onwards. However, it would also be possible if such a cleanroom started even before the heating means 30 and extended continuously to the end of the filling means, so that it is ensured that the containers are heated and moulded and also filled within the cleanroom.

Reference numeral 35 identifies sterilisation units for sterilising the plastic preforms. These sterilisation units could be arranged for example on the transport unit 22 and/or the heating unit 30. It would also be possible here for several such sterilisation units 35 to be provided. In addition, also sterilisation units for sterilising the container closure caps may be provided.

FIG. 2 shows a schematic view for illustrating possible contact surfaces. Again, a plastic preform 10 and a blow mould having two lateral parts 44 and 46 as well as a bottom part 48 are shown. Apart from that, also a transport member 8 is shown, as well as a stretching rod 6, with individual components contacting the plastic preform as a rule not at the same time, but with a time delay. The respective components 44, 46 and 48 of the blow mould have here contact surfaces 14 which contact a base body or an external wall 10b of the plastic preform 10.

Reference numeral 12 identifies a contact surface of the transport member which may come into contact with the plastic preform for example below a thread 10a thereof in order to retain the preform. The contact surfaces may each have a germicidal coating (not shown in more detail).

Reference numeral 42 also identifies in a very schematic manner a blow nozzle that correspondingly also includes a contact surface 14 by which it contacts a top edge of the plastic preform 10. The stretching rod 6 also has a contact surface 14 in particular at a bottom edge thereof, which contacts the internal wall of the plastic preform 10.

As explained above, the packagings are in particular plastic preforms. However, the subject of the disclosure would also be applicable to other packaging means such as for example plastic bottles, glass bottles, cartons, closure caps and the like.

It will be apparent to those skilled in the art that various modifications and variations can be made to the blow mould assembly of the present disclosure without departing from the scope of the invention. Throughout the disclosure, use of the terms “a,” “an,” and “the” may include one or more of the elements to which they refer. Other embodiments of the invention will be apparent to those skilled in the art from consideration of the specification and practice of the invention disclosed herein. It is intended that the specification and examples be considered as exemplary only.

What is claimed is:

1. An apparatus for treating packagings, including containers, the apparatus comprising:
at least one transport unit which transports the containers along a specified transport path, said transport unit including a transport element which contacts at least an area of the containers during transport via a first contact surface; and
at least one treatment unit which treats the containers in a specified manner, said treatment unit having a treatment element which is in contact, at least temporarily, with a surface of the container via a second contact surface,
wherein at least one of said first and second contact surfaces is designed to be germicidal.

2. The apparatus as claimed in claim 1, wherein at least one of said first and second contact surfaces has a germicidal coating.

3. The apparatus as claimed in claim 1, wherein at least one of said first and second contact surfaces has a silver ion coating.

4. The apparatus as claimed in claim 1, further comprising a cleanroom, within which the containers are transported.

5. The apparatus as claimed in claim 1, wherein the transport member is selected from a group of transport members which includes grippers, heating mandrels, and sorting rollers.

6. The apparatus as claimed in claim 1, wherein the treatment element is selected from a group of treatment elements including blow nozzles, stretching rods, blow moulds, heating mandrels, and support rings.

7. The apparatus as claimed in claim 1, further comprising a sterilisation unit that sterilises the containers at least in sections.

8. The apparatus as claimed in claim 7, wherein at least one contact of said first and second contact surfaces is arranged downstream in relation to the sterilisation unit in the transport direction of the container.

9. The apparatus as claimed in claim 1, further comprising a moulding unit for moulding plastic preforms into plastic containers.

10. The apparatus as claimed in claim 1, wherein in a specified transport area of the containers, all the contact surfaces that come into contact with the containers have been modified so as to be germicidal.

11. A method for treating packagings, including containers, the method comprising:
transporting a container with transport members of a first transport unit, the transport members being temporarily in contact with the container via a first contact surface; and
treating the container with treatment members of a treatment unit, the treatment members of the treatment unit being temporarily in contact with the container via a second contact surface,
wherein at least one of the first and second contact surfaces is designed to be germicidal.
